(19) United States
(12) Patent Application Publication
(10) Pub. No.: US 2010/0138947 A1
(43) Pub. Date: Jun. 3, 2010

(54) Method for Producing Stem Cells or Stel Cell-Like Cells from Mammalian Embryos

(76) Inventors: Ivan Vassiliev, Urrbrae (AU); Paul John Verma, Scoresby (AU)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 11/919,597

(22) PCT Filed: Apr. 28, 2006
(86) PCT No.: PCT/AU2006/000562

§ 371 Date: Jan. 14, 2010

Related U.S. Application Data
(60) Provisional application No. 60/675,857, filed on Apr. 29, 2005.

(51) Int. Cl. C12N 15/873; A61D 19/00; C12N 5/0735; A61K 45/00; A61P 39/00

(52) U.S. Cl. 800/21; 600/33; 435/325; 435/377; 424/93.7

(57) ABSTRACT

The present invention relates to methods and compositions for the production and derivation of pluripotent stem cells from embryos or embryo-derived cells and therapeutic uses therefor. In particular, the present invention relates to a method for producing functional stem cells or stem cell-like cells comprising the steps of culturing an embryo or embryo-derived cells in the presence of a demethylation agent and isolating functional pluripotent cells.

### FIELD

The present invention relates to methods and compositions for the production and derivation of pluripotent stem cells from embryos or embryo-derived cells and therapeutic uses therefor. In particular, the present invention relates to a method for producing stem cells or stem cell-like cells using a demethylation agent.

### BACKGROUND

There is an almost universal appreciation that stem cells have the potential to revolutionise biology, medical/veterinary treatments and animal husbandry. For example, numerous diseases that are the result of cell dysfunctions or the destruction of certain tissues could be treated with cell therapy. Stem cells could be induced to develop into specialised cells that after transplantation could create or contribute to new tissues. It is considered by many that such cell implants would suffer less tissue rejection than traditionally grafted tissues. Thus, human degenerative diseases such as Alzheimer's, Parkinson's, diabetes and the like could all be treated using stem cells.

However, to date the challenge has been obtaining sufficient stem cells to enable research to be undertaken and thereby provide commercially viable procedures. Currently, pluripotent cells such as stem cells have been obtained from three sources:

1). Inner cell mass cells of embryos, producing embryonic stem cells (ES cells). See for example, Thomson et al., 1998, Science, 282: p 1145; Reubinoff et al., 2000, Nature Biotechnology 18(4):399-404; U.S. Pat. No. 6,875,607

2). Foetal tissues, producing embryonic germ cells (EG cells (see, for example, Shamblott et al., 1998, P.N.A.S. USA, 95: p 13726-13731; Gearhart, 1999, Science, 282: p 1061-1062; U.S. Pat. No. 6,090,622); and

3). Umbilical cord blood. See, for example, Erices et al., 2000, British Journal of Haematology, 109 (1): 235-242; Mareschi et al., Haematologica, 86 (10): 1099-1100).

Unfortunately, the availability of the above tissues, in particular embryos and foetal tissue is very limited and likely to remain so for some time. More importantly, even if these tissues were freely available the current culturing techniques for isolating stem cells from species, other then mouse or human, are not capable of producing commercially viable quantities. Even in mice the percentage of stem cells that could be recovered from each cultured mouse blastocyst is less then 5% of the total cell number (Markert & Petters, 1978, Science, 202: 491-498). For other species this number could vary.

Accordingly, a number of researchers have proposed using other tissues to derive stem cells. For example, it has been proposed that human pluripotent stem cells can be derived via the reprogramming of somatic cell nuclei via nuclear transfer to oocytes (Munsie et al, 2000, Curr Biol, 10: p 989). Such an approach, called therapeutic cloning, would allow for pluripotent stem cells derived from a patient to be used in autologous transplant therapy (see, for example, U.S. Pat. Nos. 5,945,577 and 6,235,970). However, while these techniques are technically feasible it has been found that differentiated somatic cells and embryos cloned from somatic cells by nuclear transfer (NT) have higher levels and perturbation of DNA methylation than gametes and early embryos produced in vivo. These technical difficulties have lead to the well documented problems of premature aging and development of pathological conditions in SCNT clones (see, for example, Hill et al., 1999, Theriogenology, 51 (8):1451-1465).

In an attempt to overcome these problems researchers have tried reducing DNA methylation in donor cells before NT by treating them with chemicals such as the DNA methyl-transferase inhibitor (5-aza-2′-deoxycytidine; 5-aza-dC). However, to date there has been no dramatic improvement in cloning efficiency of NT embryos (see, for example, Jones et al., 2001, Molecular Reproduction and Development, 60: 208-213). Moreover, these embryos have not been able to provide any greater numbers of stem cells than standard culturing techniques. There is a need to develop in vitro techniques capable of producing greater numbers of pluripotent cells that do not suffer from the problems of DNA methylation and the like.

### SUMMARY

The inventors have now surprisingly found a reliable and selective process for the production of stem cells from whole embryos or embryo-derived cells.

Accordingly, in a first aspect the present invention provides a method for producing functional stem cells or stem cell-like cells comprising the steps of culturing an embryo or embryo-derived cells in the presence of a demethylation agent and isolating functional pluripotent cells.

The step of culturing the embryo or embryo-derived cells can utilise any method known in the art for culturing such cells. Preferably, the culture medium is ES cell culture medium. More preferably, the culture medium is selected from the group consisting of Synthetic Oviductal Fluid (SOF), Modified Eagle's Medium (MEM), Dulbecco's Modified Eagle's Medium (DMEM), RPMI 1640, F-12, IMDM, alpha-MEM and McCoy's Medium. Most preferably, the culture medium is alpha-MEM.

While the demethylation agent can be any agent known to demethylate DNA, it is preferably 5-azacytidine, 5-aza-2′-deoxycytidine or ethionine. Most preferably, 5-azacytidine.

It will be appreciate by those skilled in the art that the amount of demethylation agent will depend upon the specific agent used. Preferably the embryo or embryo-derived cells are incubated in the presence of about 5 μM 5-cytidine for about 10 days, to induce global genomic demethylation. These cells may also be treated with a deacetylation inhibitor or acetylation promoter, preferably 100 ng/ml or 1 μM of trichostatin A for about 24 hours, to promote histone acetylation. These cells may also be treated with an amount of a polypeptide comprising a nuclear chaperone or other chromatin remodeling enzyme, preferably nucleoplasmin or tat-nucleoplasmin, to facilitate the removal of transcription repressors from the DNA

In another embodiment, stem cells or stem cell-like cells are directly cultured under conditions that are not optimal for maintaining stem cells, but rather allow the remodeled cells to differentiate. Generally, such culture conditions may lack serum, lack feeder cells, contain a high density of cells, or contain one or more of various morphogenic growth or differentiation factors, such as retinoic acid or nerve growth factor.

The serum in the culture medium may be allogeneic serum (i.e., from the same animal species, but not the same animal), autologous serum (i.e., from the same animal) or xenogeneic serum (i.e., from a different animal species). Preferably, heat-inactivated serum, appropriate for species will be used (for example, human—autologous serum, bovine—allogenic serum, mouse—xenogeneic serum).

While the culture medium may simply be a commercially available medium like DMEM, supplemented with serum, it is appreciated that other supplements may be included. For example, growth factors, co-factors, salts and antibiotics may be included.

In a second aspect the present invention provides a method for producing stem cells or stem cell-like cells comprising:

(i) culturing an embryo or embryo-derived cells on a feeder layer of cells;

(ii) introducing to said culture at least one demethylation agent; and

(iii) isolating pluripotent cells.

Preferably, the embryo is crushed and depressed into feeder layer. In some embodiments, the feeder cell layer comprises cultured autologous cells.

In a third aspect the present invention provides an isolated stem cell or stem cell-like cell obtained by a method according to the first or second aspects.

The embryo or embryo-derived cells can be obtained from any animal, including humans. Preferably, the animal is a mammal from the one of the mammalian orders. The mammalian orders include Monotremata, Metatheria, Didelphimorphia, Paucituberculata, Microbiotheria, Dasyuromorphia, Peraamelemorphia, Notoryctemorphia, Diprotodontia, Insectivora, Macroscelidea, Scandentia, Dermoptera, Chiroptera, Primates, Xenarthra, Pholidota, Lagomorpha, Rodentia, Cetacea, Carnivora, Tubulidentata, Proboscidea, Hyracoidea, Sirenia, Perissodactyla and Artiodactyla.

Preferably, the mammal is selected from the group consisting of platypus, echidna, kangaroo, wallaby, shrews, moles, hedgehogs, tree shrews, elephant shrews, bats, primates (including chimpanzees, gorillas, orangutans, humans), edentates, sloths, armadillos, anteaters, pangolins, rabbits, picas, rodents, whales, dolphins, porpoises, carnivores, aardvark, elephants, hyraxes, dugongs, manatees, horses, rhinos, tapirs, antelope, giraffe, cows or bulls, bison, buffalo, sheep, big-horn sheep, horses, ponies, donkeys, mule, deer, elk, caribou, goat, water buffalo, camels, llama, alpaca, pigs and hippos.

In some embodiments, the embryos or embryo-derived cells are isolated from an ungulate selected from the group consisting of domestic or wild bovid, ovid, cervid, suid, equid and camelid.

Especially, preferred ungulates are Bos taurus, Bos indicus, and Bos buffalo cows or bulls.

In other embodiments, the embryos or embryo-derived cells are isolated from a human subject.

Once isolated the stem cells or stem cell-like cells of the present invention may be used in any technique that uses stem cells. For example, they can be used in a method of creating a normal non-human animal; or a method for differentiating the stem cells or stem cell-like cells ex vivo to obtain a cell, tissue or organ, or a method of treating a disease; or a method of cloning a non-human animal. Or they can be differentiated into gametes that can be used to create embryos.

Accordingly, in a fourth aspect, the present invention provides a method of creating a normal non-human animal comprising the steps of:

(a) culturing an embryo or embryo-derived cells in the presence of a demethylation agent;

(b) isolating pluripotent cells;

(c) introducing said pluripotent cells into a blastocyst;

(d) implanting the blastocyst of (c) into a surrogate mother; and

(e) allowing the offspring to develop and be born.

Preferably, the animal is chimeric.

In a fifth aspect, the present invention provides a composition comprising a population of pluripotent cells and a culture medium, wherein the pluripotent cells have been obtained by culturing an embryo or embryo-like cells in the presence of a demethylation agent.

In a sixth aspect, the present invention provides a composition comprising a population of fully or partially purified progeny of pluripotent cells according to the sixth aspect.

Preferably, the progeny have the capacity to be further differentiated. More preferably, the progeny have the capacity to terminally differentiate. Most preferably, the progeny are of the osteoblast, chondrocyte, adipocyte, fibroblast, marrow stroma, skeletal muscle, smooth muscle, cardiac muscle, occular, endothelial, epithelial, hepatic, pancreatic, hematopoietic, glial, neuronal or oligodendrocyte cell type.

In a seventh aspect, the present invention provides a method for isolating and propagating pluripotent cells comprising the steps of:

(a) obtaining an embryo or embryo-like cells from a mammal;

(b) culturing said embryo or embryo-like cells in the presence of at least one demethylation agent;

(c) recovering said pluripotent cells; and

(d) culturing said pluripotent cells under expansion conditions to produce an expanded cell population.

In an eighth aspect, the present invention provides an expanded cell population obtained by the method of the seventh aspect.

In a ninth aspect, the present invention provides a method for differentiating pluripotent cells ex vivo comprising the steps of:

(a) obtaining an embryo or embryo-like cells from a mammal;

(b) culturing said embryo or embryo-like cells in the presence of at least one demethylation agent;

(c) recovering said pluripotent cells;

(d) culturing said pluripotent cells under expansion conditions to produce an expanded cell population; and

(e) culturing the expanded cell population in the presence of desired differentiation factors.

Preferably, the differentiation factors are selected from the group consisting of basic fibroblast growth factor (bFGF); vascular endothelial growth factor (VEGF); dimethylsulfoxide (DMSO) and isoproterenol; and, fibroblast growth factor4 (FGF4) and hepatocyte growth factor (HGF).

Preferably, the differentiated cell obtained by the method of aspect nine is ectoderm, mesoderm or endoderm. More preferably, the differentiated cell is of the osteoblast, chondrocyte, adipocyte, fibroblast, marrow stroma, skeletal muscle, smooth muscle, cardiac muscle, occular, endothelial, epithelial, hepatic, pancreatic, hematopoietic, glial, neuronal or oligodendrocyte cell type.

In a tenth aspect, the present invention provides a method for differentiating pluripotent cells in vivo comprising the steps of:

(a) obtaining an embryo or embryo-like cells from a mammal;

(b) culturing said embryo or embryo-like cells in the presence of at least one demethylation agent;

(c) recovering said pluripotent cells;

(d) culturing said pluripotent cells to produce an expanded cell population; and

(e) administering the expanded cell population to a mammalian host, wherein said cell population is engrafted and differentiated in vivo in tissue specific cells, such that the function of a cell or organ, defective due to injury, genetic disease, acquired disease or iatrogenic treatments, is augmented, reconstituted or provided for the first time.

Preferably, the tissue specific cells are of the osteoblast, chondrocyte, adipocyte, fibroblast, marrow stroma, skeletal muscle, smooth muscle, cardiac muscle, occular, endothelial, epithelial, hepatic, pancreatic, hematopoietic, glial, neuronal or oligodendrocyte cell type.

Preferably, the disease is selected from the group consisting of cancer, cardiovascular disease, metabolic disease, liver disease, diabetes, hepatitis, hemophilia, degenerative or traumatic neurological conditions, autoimmune disease, genetic deficiency, connective tissue disorders, anemia, infectious disease and transplant rejection.

In an eleventh aspect, the present invention provides a therapeutic composition comprising pluripotent cells and a pharmaceutically acceptable carrier, wherein the pluripotent cells are present in an amount effective to produce tissue selected from the group consisting of bone marrow, blood, spleen, liver, lung, intestinal tract, eye, brain, immune system, bone, connective tissue, muscle, heart, blood vessels, pancreas, central nervous system, kidney, bladder, skin, epithelial appendages, breast-mammary glands, fat tissue, and mucosal surfaces including oral esophageal, vaginal and anal and wherein said pluripotent cells are produced by culturing an embryo or embryo-derived cells in the presence of at least one demethylation agent.

In a twelfth aspect, the present invention provides a therapeutic method for restoring organ, tissue or cellular function to a mammalian animal in need thereof comprising the steps of:

(a) obtaining an embryo or embryo-like cells from a mammal;

(b) culturing said embryo or embryo-like cells in the presence of at least one demethylation agent;

(c) recovering said pluripotent cells; and

(d) administering the pluripotent cells to the mammalian animal, wherein organ, tissue or cellular function is restored.

A thirteenth aspect provides a method of nuclear transfer comprising the step of transferring a pluripotent cell obtained by culturing an embryo or embryo-derived cells in the presence of at least one demethylation agent or a nuclei isolated therefrom into an enucleated oocyte.

A fourteenth aspect provides a method for producing a genetically engineered or transgenic non-human mammal comprising:

(i) inserting, removing or modifying a desired gene into a pluripotent cell obtained by culturing an embryo or embryo-derived cells in the presence of at least one demethylation agent or a nuclei isolated therefrom; and

(ii) transferring the pluripotent cell or nuclei into an enucleated oocyte.

A fifteenth aspect provides a method for cloning a non-human mammal comprising:

(i) inserting a pluripotent cell obtained by culturing an embryo or embryo-derived cells in the presence of at least one demethylation agent or a nuclei isolated therefrom into an enucleated mammalian oocyte, under conditions suitable for the formation of a reconstituted cell;

(ii) activating the reconstituted cell to form an embryo;

(iii) culturing said embryo until greater than the 2-cell developmental stage; and

(iv) transferring said cultured embryo to a host mammal such that the embryo develops into a fetus.

Oocytes may be isolated from any non-human mammal by known procedures. For example, oocytes can be isolated from either oviducts and/or ovaries of live animals by oviductal recovery procedures or transvaginal oocyte recovery procedures well known in the art and described herein. Furthermore, oocytes can be isolated from deceased animals. For example, ovaries can be obtained from abattoirs and the oocytes aspirated from these ovaries. The oocytes can also be isolated from the ovaries of a recently sacrificed animal or when the ovary has been frozen and/or thawed. Preferably, the oocytes are freshly isolated from the oviducts.

Also provided by the present invention are non-human mammals obtained according to the above methods, and offspring of those mammals.

In a sixteenth aspect the present invention provides the use of cells of the present invention to deliver vaccines, RNAi vectors, transgenes, DNA vectors, ectopically to specific sites.

Use of the cells of the present invention for gene therapy applications are also envisaged.

### BRIEF DESCRIPTION OF THE FIGURES

FIG. 1 shows embryo's squashed and depressed into feeder layer. Control embryo (Panel A) and test embryo, which will be subjected to 5-azacytidine treatment (Panel B), are quite similar in appearance.

FIG. 2 (Panel A) shows control outgrowth (CO) after 7 to 9 days in vitro culture consists of several types of cells: pluripotent cells(PC) with morphology, characteristic for bovine ES cells; trophoblast cells; primitive endoderm cells. Panel B shows that treated embryo develops outgrowth (treated outgrowth, TRO) consisting only from cell with morphology characteristic for bovine ES cells.

FIG. 3 shows pluripotent cells of CO express markers of pluripotency: bovine Oct4, bovine Rex1 and bovine SSEA-1 (Panel A). Other cells of CO do not express these markers (Panel B). Cells of any randomly chosen region of TRO express Oct4, Rex1 (Panel C) and SSEA-1 (Panel D).

FIG. 4 shows that after 21 days in culture the confluent TRO express Oct4.

FIG. 5 shows just passaged TRO.

FIG. 6 shows passaged TRO after 2 days of culture in presence of 5-azacytidine.

FIG. 7 shows passaged TRO after 7 days in culture in presence of 5-azacytidine.

FIG. 8 (Panel A) shows dilated epithelial gland (glandular epithelium) lined by a single layer of cuboidal to columnar epithelium, the later has eosinophilic, pink, apical cytoplasm and basale located uniformly sized oval nuclei. At the upper right border of the picture there is an erythrocyte filled blood vessels. Panel B shows that ectoderm derivatives are presented by small dark neuroblastic cells (neuronal differentiation). Panel C shows epithelial gland, consisting of cuboidal cells with esoinophilic, pink cytoplasm and uniformly sized oval nuclei, containing amorphous eosinophilic proteinaceous secretion (endoderm). Gland is surrounded by loosely packed collagen fibres (mesoderm).

FIG. 9 Panels A to E show mouse embryos cultured under various conditions.

### DETAILED DESCRIPTION OF PREFERRED EMBODIMENTS

Before describing the present invention in detail, it is to be understood that this invention is not limited to particularly exemplified cell culture techniques, serum, media or methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols, reagents and vectors which are reported in the publications and which might be used in connection with the invention. Nothing herein is to be construed as an admission that the invention is not entitled to antedate such disclosure by virtue of prior invention.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

It must be noted that as used herein and in the appended claims, the singular forms “a,” “an,” and “the” include plural reference unless the context clearly dictates otherwise. Thus, for example, a reference to “a cell” includes a plurality of such cells, and a reference to “an oocyte” is a reference to one or more oocytes, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The present invention relates to methods of producing functional stem cells or stem cell-like cells from intact embryo's or embryo-derived cells. As used herein, the term “embryo” or “embryonic” refers to a developing cell mass that has not implanted into a uterine membrane of a maternal host. Hence, the term “embryo” as used herein is defined as any stage after fertilization up to 8 weeks post conception. It develops from repeated division of cells and includes the pre-blastocyst stage, the blastocyst stage, and/or any other developing cell mass stages that develop prior to implantation into a uterine membrane of a maternal host. Embryos of the “blastocyst stage” comprise an outer trophectoderm and an inner cell mass (ICM). The term “embryo-derived cell” includes any cell or groups of cells isolated from and/or arisen from an embryo. In some embodiments, the term “embryo-derived cell” includes any number of cells associated with embryo's including trophectoderm cells.

An embryo can represent multiple stages of cell development. For example, a one cell embryo can be referred to as a zygote, a solid spherical mass of cells resulting from a cleaved embryo can be referred to as a morula, and an embryo having a blastocoel can be referred to as a blastocyst.

The embryos or embryo-derived cells may be taken from any animal, for which the study of stem cells or stem cell-like cells is required. Suitable mammalian animals include members of the Orders Primates, Rodentia, Lagomorpha, Cetacea, Carnivora, Perissodactyla and Artiodactyla. Members of the Orders Perissodactyla and Artiodactyla are particularly preferred because of their similar biology and economic importance.

For example, Artiodactyla comprise approximately 150 living species distributed through nine families: pigs (Suidae), peccaries (Tayassuidae), hippopotamuses (Hippopotamidae), camels (Camelidae), chevrotains (Tragulidae), giraffes and okapi (Giraffidae), deer (Cervidae), pronghorn (Antilocapridae), and cattle, sheep, goats and antelope (Bovidae). Many of these animals are used as feed animals in various countries. More importantly, with respect to the present invention, many of the economically important animals such as goats, sheep, cattle and pigs have very similar biology and share high degrees of genomic homology.

The Order Perissodactyla comprises horses and donkeys, which are both economically important and closely related. Indeed, it is well known that horses and donkeys interbreed.

In some embodiments, the embryo or embryo-derived cells will be obtained from ungulates; and in particular, bovids, ovids, cervids, suids, equids and camelids. Examples of such representatives are cows or bulls, bison, buffalo, sheep, big-horn sheep, horses, ponies, donkeys, mule, deer, elk, caribou, goat, water buffalo, camels, llama, alpaca, and pigs. Especially preferred bovine species are Bos taurus, Bos indicus, and Bos buffaloes cows or bulls.

In other embodiments, the embryo or embryo-derived cells will be obtained from primates, especially humans.

Once the embryo or embryo-derived cells have been obtained they are then cultured. The general purpose of the culture is to “isolate,” “proliferate” or “selectively expand” functional stem cells or stem cell-like cells present in the embryo or embryo-derived cells. The terms “isolate,” “proliferate” or “selectively expand” as used herein refers to the culturing process by which the stem cells or stem cell-like cells are increased in number relative to the other cells present in the embryo or embryo-derived cells.

The term “progenitor cell” is used synonymously with “stem cell”. Both terms refer to an undifferentiated cell which is capable of proliferation and giving rise to more progenitor cells having the ability to generate a large number of mother cells that can in turn give rise to differentiated or differentiable daughter cells. In preferred embodiments, the term progenitor or stem cell refers to embryonic stem cells or stem cell-like cells (ES cells). The characteristics of ES cells are loss of contact inhibition, anchorage independent growth, de novo expression of alkaline phosphatase and activation of the germ line specific Oct4 promoter. Accordingly, the term “functional” as used herein refers to the ability of the stem cells of the present invention, which have been isolated from embryos to exhibit at least one of the following activities: express SSEA-1, SSEA-3, SSEA-4, TRA 1-60, TRA 1-81, GCTM-2, alkaline phosphatase, Oct-4, Rex1, Nanong; grow as flat colonies, monolayer colonies or colonies, growing as clumps of cells with distinct cell borders; differentiate into derivatives of all three embryonic germ layers; and unresponsive to Leukemia Inhibitory Factor (LIF). (See, for example, Pera et al., 1989, Differentiation, 42: p 10-23).

The terms “culture,” “cultured” and “culturing” are used herein interchangeably, to refer to the process by which the embryo and/or embryo-derived cells are grown in vitro.

The embryo may be subjected to physical and/or chemical dissociating means capable of dissociating cellular stratum. Methods for dissociating cellular layers within the embryo are well known in the field. For example, the dissociating means may be either a physical or a chemical disruption means. Physical dissociation means might include, for example, scraping the embryo with a scalpel, mincing the embryo, physically cutting the embryo apart, or perfusing the embryo with enzymes. Chemical dissociation means might include, for example, digestion with enzymes such as trypsin, dispase, collagenase, trypsin-EDTA, thermolysin, pronase, hyaluronidase, elastase, papain and pancreatin. Non-enzymatic solutions for the dissociation of the embryo can also be used.

The dissociation of the embryo can be achieved by placing the embryo in a pre-warmed enzyme solution containing an amount of trypsin sufficient to dissociate the cellular stratum in the embryo. Preferably, the enzyme solution used in the method is calcium and magnesium free.

The amount of trypsin that might be used in the method is preferably between about 5 and 0.1% trypsin per volume of solution. Desirable the trypsin concentration of the solution is about 2.5 to 0.25%, with about 0.5% trypsin being most preferred.

The time period over which the embryo is subjected to the trypsin solution may vary depending on the size of the embryo. Preferably the embryo is placed in the presence of the trypsin solution for sufficient time to weaken the cohesive bonding between the cells of the embryo. For example, the embryo might be placed in trypsin for between 5 to 6.0 minutes. In some embodiments, the embryo is immersed in the trypsin solution for between 10 and 30 minutes with 15 to 20 minutes being optimal for most embryos.

However, in some preferred embodiments, the embryo is left intact and merely introduced into tissue culture medium. The terms “culture media,” “tissue culture media” or “tissue culture medium” are recognised in the art, and refers generally to any substance or preparation used for the cultivation of living cells. There are a large number of tissue culture media that exist for culturing tissue from animals. Some of these are complex and some are simple. Examples of media that would be useful in the present invention include Modified Eagle's Medium (MEM), Dulbecco's Modified Eagle's Medium (DMEM), RPMI 1640, F-12, IMDM, alpha-MEM and McCoy's Medium. Most preferably, the culture medium is alpha-MEM.

However in some preferred embodiments, intact embryos are squashed and depressed into feeder layer on the dish using fine-tipped glass micropipettes or Ultra-fine needle insulin syringe. Without wishing to be bound by any theory or hypothesis the inventors believe that all previously used methods of isolating ES cells from embryos involved the mechanical, immunosurgical or enzymatic isolation of ICM or ICM derivatives from other cells of the embryo. In contrast, the methods of the present invention allow the use of whole embryos, wherein all cells of a pre-implantation embryo are converted into a pluripotent state.

In some embodiments, the embryo or embryo-derived cells are introduced into alpha-MEM supplemented with 2 mM glutamax, 1% non-essential amino acids, 0.1 mM mercaptoethanol, 100 U/ml penicillin, and 100 mg/ml streptomycin, 1.25 μg/ml amphotericin B, 5 ng/ml bFGF, 5 ng/ml hEGF, 1×ITS solution, 5 ng/ml hLIF (all from Invitrogen).

In order to encourage the stem cells or stem cell-like cells to proliferate, serum is added to the tissue culture medium. The serum in the culture medium may be allogeneic serum (i.e., from the same animal species, but not the same animal), autologous serum (i.e., from the same animal) or xenogeneic serum (i.e., from a different animal species). In some embodiments, heat-inactivated autologous serum is used.

When the embryo or embryo-derived cells are initially cultured the amount of serum used is typically about 20%. The term “about” as used herein to describe the amount of serum used in the culture medium indicates that in certain circumstances the amount of serum used will be slightly more (approximately 10% more) or slightly less (approximately 10% less), than the stated amount. For example, about 10% serum would mean that as little as 18% serum might be used or up to a maximum of 22% serum.

The embryo or embryo-derived cells, including the stem cells or stem cell-like cells are incubated in a humidified 95% air/5% CO₂ atmosphere. The temperature of incubation is in appropriate conditions for different species. For example, for mouse and human temperature of incubation is 37° C.; for bovine temperature of incubation is 39° C.

The media is also supplemented with at least one demethylation agent. The term “demethylation agent” as used herein includes inhibitors of DNA, methyltransferases or inhibitors of histone deacetylase, or inhibitors of a repressor complex.

Presently preferred demethylation agents comprise at least one of 5-azacytidine, 5-aza-2′-deoxycytidine, 2-amino-4-(ethylthio)butyric acid, procainamide, procaine, Ara-C, decitabine, fazarabine, DHAC.

Without wishing to be bound by any theory or hypothesis the inventors believe that the presence of the demethylation agent assists in the isolation of stem cells or stem cell-like cells from the embryo or embryo-derived cells. Indeed, in some embodiments, the stem cells isolated by the methods of the present invention comprise more than 90% of the cells present in culture.

The amount of demethylation agent will depend on the type of agent used, the volume of cells, type of media and/or the number of embryos. In some embodiments, the demethylation agent is 5-azacytidine at a concentration of less than 30 μM, more preferably between 0.01 and 20 μM and most preferably about 5 μM.

In some embodiments, the embryo or embryo-derived cells of the present invention are cultured on a feeder layer. Examples of feeder layers are well known to a person of ordinary skill in the art, and can arise from a number of different cells that are cultured in vitro. See, e.g., exemplary embodiment described hereafter and Strelchenko, 1996, Theriogenology 45: 130-141; Piedrahita et al., 1990, Theriogenology 34: 879-901; Piedrahita et al., 1998, Biol. Reprod. 58: 1321-1329; and Shim et al., 1997, Theriogenology 47: 245, each of which is incorporated herein by reference in its entirety including all figures, tables, and drawings.

As the stem cells or stem cell-like cells proliferate they, depending upon species, generally produce colonies with a flattened appearance (human), monolayer colonies appearance (bovine), cell clumps colonies appearance (mouse). Once colonies reach approximately 0.5 cm in diameter they can be mechanically cut into several pieces and manually replated onto fresh feeder layer in fresh medium with demethylation agent such as 5-azacytidine.

Once the stem cells and/or stem cell-like cells have been isolated or proliferated they can then be used, for example, for direct transplantation or to produce differentiated cells in vitro for transplantation or in nuclear transfer techniques. The invention accordingly provides, for example, stem cells that may serve as a source for many other, more differentiated cell types.

One embodiment pertains to the progeny of the stem cells and/or stem cell-like cells e.g. those cells which have been derived from the cells of the initial embryo. Such progeny can include subsequent generations of stem cells and/or stem cell-like cells as well as lineage committed cells generated by inducing differentiation of the stem cells and/or stem cell-like cells after their isolation from the embryos, e.g., induced in vitro.

Another embodiment relates to cellular compositions enriched for stem cells and/or stem cell-like cells, or the progeny thereof. In certain embodiments, the cells will be provided as part of a pharmaceutical preparation, e.g., a sterile composition, free of the presence of unwanted virus, bacteria and other pathogens, as well as pyrogen-free preparation. That is, for animal administration, the stem cells and/or stem cell-like cells should meet sterility, pyrogenicity, general safety and purity standards as required by FDA Office of Biologics standards.

In certain embodiments, such cellular compositions can be used for transplantation into animals, preferably mammals, and even more preferably humans. The stem cells and/or stem cell-like cells can be autologous, allogeneic or xenogeneic with respect to the transplantation host.

Yet another aspect of the present invention concerns cellular compositions, which include as a cellular component, substantially pure preparations of the stem cells and/or stem cell-like cells, or the progeny thereof. Cellular compositions of the present invention include not only substantially pure populations of the stem cells and/or stem cell-like cells, but can also include cell culture components, e.g., culture media including amino acids, metals, coenzyme factors, as well as small populations of non-stem cells or stem cell-like cells, e.g., some of which may arise by subsequent differentiation of isolated stem cells and/or stem cell-like cells of the invention. Furthermore, other non-cellular components include those which render the cellular component suitable for support under particular circumstances, e.g., implantation, e.g., continuous culture.

As common methods of administering the stem cells and/or stem cell-like cells of the present invention to animals, particularly humans, which are described in detail herein, include injection or implantation of the stem cells and/or stem cell-like cells into target sites in the animals, the cells of the invention can be inserted into a delivery device which facilitates introduction by, injection or implantation, of the cells into the animals. Such delivery devices include tubes, e.g., catheters, for injecting cells and fluids into the body of a recipient animal. In a preferred embodiment, the tubes additionally have a needle, e.g., a syringe, through which the cells of the invention can be introduced into the animal at a desired location. The stem cells and/or stem cell-like cells of the invention can be inserted into such a delivery device, e.g., a syringe, in different forms. For example, the cells can be suspended in a solution or embedded in a support matrix when contained in such a delivery device. As used herein, the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating stem cells and/or stem cell-like cells as described herein in a pharmaceutically acceptable carrier or diluent and, as required, other ingredients enumerated above, followed by filtered sterilisation.

Support matrices in which the stem cells and/or stem cell-like cells can be incorporated or embedded include matrices which are recipient-compatible and which degrade into products which are not harmful to the recipient. Natural and/or synthetic biodegradable matrices are examples of such matrices. Natural biodegradable matrices include plasma clots, e.g., derived from a mammal, and collagen matrices. Synthetic biodegradable matrices include synthetic polymers such as polyanhydrides, polyorthoesters, and polylactic acid. Other examples of synthetic polymers and methods of incorporating or embedding cells into these matrices are known in the art. See e.g., U.S. Pat. Nos. 4,298,002 and 5,308,701. These matrices provide support and protection for the fragile progenitor cells in vivo and are, therefore, the preferred form in which the stem cells and/or stem cell-like cells are introduced into the recipient animals.

The present invention also provides substantially pure stem cells and/or stem cell-like cells which can be used therapeutically for treatment of various disorders.

To illustrate, the stem cells and/or stem cell-like cells of the invention can be used in the treatment or prophylaxis of a variety of disorders. For instance, the stem cells and/or stem cell-like cells can be used to produce populations of differentiated cells for repair of damaged tissue e.g. pancreatic tissue, cardiac tissue, nerves and the like. Likewise, such cell populations can be used to regenerate or replace pancreatic tissue, cardiac tissue or nerves lost due to, pancreatolysis, e.g., destruction of pancreatic tissue, such as pancreatitis, heart disease or neuropathy.

Yet another embodiment provides methods for screening various compounds for their ability to modulate growth, proliferation or differentiation of stem cells and/or stem cell-like cells. In an illustrative embodiment, the subject stem cells and/or stem cell-like cells, and their progeny, can be used to screen various compounds or natural products. Such explants can be maintained in minimal culture media for extended periods of time (e.g., for 7-21 days or longer) and can be contacted with any compound, e.g., small molecule or natural product, e.g., growth factor, to determine the effect of such compound on one of cellular growth, proliferation or differentiation of the stem cells and/or stem cell-like cells. Detection and quantification of growth, proliferation or differentiation of these cells in response to a given compound provides a means for determining the compound's efficacy at inducing one of the growth, proliferation or differentiation. Methods of measuring cell proliferation are well known in the art and most commonly include determining DNA synthesis characteristic of cell replication. There are numerous methods in the art for measuring DNA synthesis, any of which may be used according to the invention. In an embodiment of the invention, DNA synthesis has been determined using a radioactive label (³H-thymidine) or labelled nucleotide analogues (BrdU) for detection by immunofluorescence. The efficacy of the compound can be assessed by generating dose response curves from data obtained using various concentrations of the compound. A control assay can also be performed to provide a baseline for comparison. Identification of the progenitor cell population(s) amplified in response to a given test agent can be carried out according to such phenotyping as described above.

In some embodiments, the stem cells and/or stem cell-like cells are used for cloning mammals by nuclear transfer or nuclear transplantation. In the subject application, the terms “nuclear transfer” or “nuclear transplantation” are used interchangeably; however, these terms as used herein refers to introducing a full complement of nuclear DNA from one cell to an enucleated cell.

The first step in the preferred methods involves the isolation of a recipient oocyte from a suitable animal. In this regard, the oocyte may be obtained from any animal source and at any stage of maturation. Methods for isolation of oocytes are well known in the art. For example, oocytes can be isolated from either oviducts and/or ovaries of live animals by oviductal recovery procedures or transvaginal oocyte recovery procedures well known in the art. See, e.g., Pieterse et al., 1988, “Aspiration of bovine oocytes during transvaginal ultrasound scanning of the ovaries,” Theriogenology 30: 751-762. Furthermore, oocytes can be isolated from ovaries or oviducts of deceased animals. For example, ovaries can be obtained from abattoirs and the oocytes aspirated from these ovaries. The oocytes can also be isolated from the ovaries of a recently sacrificed animal or when the ovary has been frozen and/or thawed.

Briefly, in one preferred embodiment, immature (prophase I) oocytes from mammalian ovaries are harvested by aspiration. For the successful use of techniques such as genetic engineering, nuclear transfer and cloning, once these oocytes have been harvested they must generally be matured in vitro before these cells may be used as recipient cells for nuclear transfer.

The stage of maturation of the oocyte at enucleation and nuclear transfer has been reported to be significant to the success of nuclear transfer methods. (See e.g., Prather et al., 1991, Differentiation, 48, 1-8). In general, successful mammalian embryo cloning practices use the metaphase II stage oocyte as the recipient oocyte because at this stage it is believed that the oocyte can be or is sufficiently activated to treat the introduced nucleus as it does a fertilising sperm.

The in vitro maturation of oocytes usually takes place in a maturation medium until the oocyte has extruded the first polar body, or until the oocyte has attained the metaphase II stage. In domestic animals, and especially cattle, the oocyte maturation period generally ranges from about 16-52 hours, preferably about 28-42 hours and more preferably about 18-24 hours post-aspiration. For purposes of the present invention, this period of time is known as the “maturation period.”

Oocytes can be matured in a variety ways and using a variety of media well known to a person of ordinary skill in the art. See, e.g., U.S. Pat. No. 5,057,420; Saito et al., 1992, Roux's Arch. Dev. Biol. 201: 134-141 for bovine organisms and Wells et al., 1997, Biol. Repr. 57: 385-393 for ovine organisms and WO97/07668, entitled “Unactivated Oocytes as Cytoplast Recipients for Nuclear Transfer,” all hereby incorporated herein by reference in the entirety, including all figures, tables, and drawings.

One of the most common media used for the collection and maturation of oocytes is TCM-199, and 1 to 20% serum supplement including FCS, newborn serum, estrual cow serum, lamb serum or steer serum. Oocytes can be successfully matured in this type of medium within an environment comprising 5% CO₂ at 39° C.

While it will be appreciated by those skilled in the art that freshly isolated and matured oocytes are preferred, it will also be appreciated that it is possible to cryopreserve the oocytes after harvesting or after maturation. Accordingly, the term “cryopreserving” as used herein can refer to freezing an oocyte, a cell, embryo, or animal of the invention. The oocytes, cells, embryos, or portions of animals of the invention are frozen at temperatures preferably lower than 0° C., more preferably lower than −80° C., and most preferably at temperatures lower than −196° C. Oocytes, cells and embryos in the invention can be cryopreserved for an indefinite amount of time. It is known that biological materials can be cryopreserved for more than fifty years. For example, semen that is cryopreserved for more than fifty years can be utilised to artificially inseminate a female bovine animal. Methods and tools for cryopreservation are well known to those skilled in the art. See, e.g., U.S. Pat. No. 5,160,312, entitled “Cryopreservation Process for Direct Transfer of Embryos”.

If cyropreserved oocytes are utilised then these must be initially thawed before placing the oocytes in maturation medium. Methods of thawing cryopreserved materials such that they are active after the thawing process are well-known to those of ordinary skill in the art.

In a further preferred embodiment, mature (metaphase II) oocytes, which have been matured in vivo, are harvested and used in the nuclear transfer methods disclosed herein. Essentially, mature metaphase II oocytes are collected surgically from either non-superovulated or superovulated cows or heifers 35 to 48 hours past the onset of estrus or past the injection of human chorionic gonadotropin (hCG) or similar hormone.

Where oocytes have been cultured in vitro cumulus cells that may have accumulated may be removed to provide oocytes that are at a more suitable stage of maturation for enucleation. Cumulus cells may be removed by pipetting or vortexing, for example, in the presence of 0.5% hyaluronidase.

After the maturation period as described above the zona pellucida may be removed from the oocytes if desired. The advantages of zona pellucida removal are described in PCT/AU02/00491, which is incorporated in its entirety herein by reference. The removal of the zona pellucida from the oocyte may be carried out by any method known in the art including physical manipulation (mechanical opening), chemical treatment or enzymatic digestion (Wells & Powell, 2000). Physical manipulation may involve the use of a micropipette or a microsurgical blade. Preferably, enzymatic digestion is used.

In one particularly preferred embodiment, the zona pellucida is removed by enzymatic digestion in the presence of a protease or pronase. Briefly, mature oocytes are placed into a solution comprising a protease, pronase or combination of each at a total concentration in the range of 0.1%-5%, more preferably 0.25%-2% and most preferably about 0.5%. The mature oocyte is then allowed to incubate at between 30° C. to about 45° C., preferably about 39° C. for a period of 1 to 30 minutes. Preferably the oocytes are exposed to the enzyme for about 5 minutes. Although pronase may be harmful to the membranes of oocytes, this effect may be minimised by addition of serum such as FCS or cow serum. The unique advantage of zona removal with pronase is that no individual treatment is required, and the procedure can be performed in quantities of 100's of oocytes. Once the zona pellucida has been removed the zona pellucida-free mature oocyte are rinsed in 4 ml HEPES buffered TCM-199 medium supplemented with 20% FCS and 10 μg/ml cytochalasin B and then enucleated.

The terms “enucleation”, “enucleated” and “enucleated oocyte” are used interchangeably herein and refers to an oocyte which has had part of its contents removed.

Enucleation of the oocyte may be achieved physically, by actual removal of the nucleus, pronuclei or metaphase plate (depending on the oocyte), or functionally, such as by the application of ultraviolet radiation or another enucleating influence. All of these methods are well known to those of ordinary skill in the art. For example, physical means includes aspiration (Smith & Wilmut, 1989, Biol. Reprod., 40: 1027-1035); functional means include use of DNA-specific fluorochromes (See, for example, Tsunoda et al., 1988, J. Reprod. Fertil. 82: 173), and irradiation with ultraviolet light (See, for example, Gurdon, 1960, J. Microsc. Soc., 101: 299-311). Enucleation may also be effected by other methods known in the art. See, for example, U.S. Pat. No. 4,994,384; U.S. Pat. No. 5,057,420; and Willadsen, 1986, Nature, 320:63-65, herein incorporated by reference.

Preferably, the oocyte is enucleated by means of manual bisection. Oocyte bisection may be carried out by any method known to those skilled in the art. In one preferred embodiment, the bisection is carried out using a microsurgical blade as described in International Patent Application. No. WO98/29532 which is incorporated by reference herein. Briefly, oocytes are split asymmetrically into fragments representing approximately 30% and 70% of the total oocyte volume using an ultra sharp splitting blade (AB Technology, Pullman, Wash., USA). The oocytes may then be screened to identify those of which have been successfully enucleated. This screening may be effected by staining the oocytes with 1 microgram per millilitre of the Hoechst fluorochrome 33342 dissolved in TCM-199 media supplemented with 20% FCS, and then viewing the oocytes under ultraviolet irradiation with an inverted microscope for less than 10 seconds. The oocytes that have been successfully enucleated (demi-oocytes) can then be placed in a suitable culture medium, e.g., TCM-199 media supplemented with 20% FCS.

In the present invention, the recipient oocytes will preferably be enucleated at a time ranging from about 10 hours to about 40 hours after the initiation of in vitro maturation, more preferably from about 16 hours to about 24 hours after initiation of in vitro maturation, and most preferably about 16-18 hours after initiation of in vitro maturation.

The bisection technique described herein requires much less time and skill than other methods of enucleation and the subsequent selection by staining results in high accuracy. Consequently, for large-scale application of cloning technology the present bisection technique can be more efficient than other techniques.

A single stem cell or stem cell-like cell of the present invention of the same species as the enucleated oocyte can then be transferred by fusion into the enucleated oocyte thereby producing a reconstituted cell.

Analysis of cell cycle stage may be performed as described in Kubota et al., PNAS 97: 990-995 (2000). Briefly, cell cultures at different passages are grown to confluency. After trypsinisation, cells are washed with TCM-199 plus 10% FCS and re-suspended to a concentration of 5×10⁵ cells/ml in 1 ml PBS with glucose (6.1 mM) at 4° C. Cells are fixed overnight by adding 3 ml of ice-cold ethanol. For nuclear staining, cells are then pelleted, washed with PBS and re-suspended in PBS containing 30 μg/ml propidium iodide and 0.3 mg/ml RNase A. Cells are allowed to incubate for 1 h at room temperature in the dark before filtered through a 30 μm mesh. Cells are then analyzed.

To examine the ploidy of the stem cells and/or stem cell-like cells at various passages, chromosome counts may be determined at different passages of culture using standard preparation of metaphase spreads (See, for example, Kubota et al., 2000, PNAS, 97: 990-995).

Cultured stem cells and/or stem cell-like cells may also be genetically altered by transgenic methods well-known to those of ordinary skill in the art. See, for example, Molecular Cloning a Laboratory Manual, 2nd Ed., 1989, Sambrook, Fritsch and Maniatis, Cold Spring Harbor Laboratory Press; U.S. Pat. No. 5,612,205; U.S. Pat. No. 5,633,067; EPO 264 166, entitled “Transgenic Animals Secreting Desired Proteins Into Milk”; WO94/19935, entitled “Isolation of Components of Interest From Milk”; WO93/22432, entitled “Method for Identifying Transgenic Pre-implantation Embryos”; and WO95/175085, entitled “Transgenic Production of Antibodies in Milk,” all of which are incorporated by reference herein in their entirety including all figures, drawings and tables. Any known method for inserting, deleting or modifying a desired gene from a mammalian cell may be used for altering the stem cells and/or stem cell-like cells to be used as the nuclear donor. These procedures may remove all or part of a gene and the gene may be heterologous. Included is the technique of homologous recombination, which allows the insertion, deletion or modification of a gene or genes at a specific site or sites in the cell genome.

Examples for modifying a target DNA genome by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

The present invention can thus be used to provide adult mammals with desired genotypes. Multiplication of adult ungulates with proven genetic superiority or other desirable traits is particularly useful, including transgenic or genetically engineered animals, and chimeric animals. Furthermore, cell and tissues from the nuclear transfer foetus, including transgenic and/or chimeric foetuses, can be used in cell, tissue and organ transplantation.

Methods for generating transgenic cells typically include the steps of (1) assembling a suitable DNA construct useful for inserting a specific DNA sequence into the nuclear genome of stem cells and/or stem cell-like cells; (2) transfecting the DNA construct into the stem cells and/or stem cell-like cells; (3) allowing random insertion and/or homologous recombination to occur. The modification resulting from this process may be the insertion of a suitable DNA construct(s) into the target genome; deletion of DNA from the target genome; and/or mutation of the target genome.

DNA constructs can comprise a gene of interest as well as a variety of elements including regulatory promoters, insulators, enhancers, and repressors as well as elements for ribosomal binding to the RNA transcribed from the DNA construct.

DNA constructs can also encode ribozymes and anti-sense DNA and/or PNA, identified previously herein. These examples are well known to a person of ordinary skill in the art and are not meant to be limiting.

Due to the effective recombinant DNA techniques available in conjunction with DNA sequences for regulatory elements and genes readily available in data bases and the commercial sector, a person of ordinary skill in the art can readily generate a DNA construct appropriate for establishing transgenic cells using the materials and methods described herein.

Transfection techniques are well known to a person of ordinary skill in the art and materials and methods for carrying out transfection of DNA constructs into cells are commercially available. Materials typically used to transfect cells with DNA constructs are lipophilic compounds, such as Lipofectin™ for example. Particular lipophilic compounds can be induced to form liposomes for mediating transfection of the DNA construct into the cells.

Target sequences from the DNA construct can be inserted into specific regions of the nuclear genome by rational design of the DNA construct. These design techniques and methods are well known to a person of ordinary skill in the art. See, for example, U.S. Pat. No. 5,633,067; U.S. Pat. No. 5,612,205 and PCT publication WO93/22432, all of which are incorporated by reference herein in their entirety. Once the desired DNA sequence is inserted into the nuclear genome, the location of the insertion region as well as the frequency with which the desired DNA sequence has inserted into the nuclear genome can be identified by methods well known to those skilled in the art.

Once the transgene is inserted into the nuclear genome of the donor stem cells and/or stem cell-like cells, that cell, like other donor stem cells and/or stem cell-like cells of the invention, can be used as a nuclear donor in nuclear transfer methods. The means of transferring the nucleus of a stem cells and/or stem cell-like cells into the enucleated oocyte preferably involves cell fusion to form a reconstituted cell.

Fusion is typically induced by application of a DC electrical pulse across the contact/fusion plane, but additional AC current may be used to assist alignment of donor and recipient cells. Electrofusion produces a pulse of electricity that is sufficient to cause a transient breakdown of the plasma membrane and which is short enough that the membrane reforms rapidly. Thus, if two adjacent membranes are induced to breakdown and upon reformation the lipid bilayers intermingle, small channels will open between the two cells. Due to the thermodynamic instability of such a small opening, it enlarges until the two cells become one. Reference is made to U.S. Pat. No. 4,997,384 by Prather et al., (incorporated by reference in its entirety herein) for a further discussion of this process. A variety of electrofusion media can be used including e.g., sucrose, mannitol, sorbitol and phosphate buffered solution.

Fusion can also be accomplished using Sendai virus as a fusogenic agent (Graham, 1969, Wister Inot. Symp. Monogr., 9, 19). Fusion may also be induced by exposure of the cells to fusion-promoting chemicals, such as polyethylene glycol.

Preferably, the donor stem cells and/or stem cell-like cells and enucleated oocyte are placed in a 500 μm fusion chamber and covered with 4 ml of 26° C.-27° C. fusion medium (0.3M mannitol, 0.1 mM MgSO₄, 0.05 mM CaCl₂). The cells are then electrofused by application of a double direct current (DC) electrical pulse of 70-100V for about 15 μs, approximately 1 s apart. After fusion, the resultant fused reconstituted cells are then placed in a suitable medium until activation, e.g., TCM-199 medium.

In a preferred method of cell fusion the donor tissue-specific progenitor cell, stem cell-like cell or MCT is firstly attached to the enucleated oocyte. For example, a compound is selected to attach the progenitor cell, stem cell-like cell or MCT to the enucleated oocyte to enable fusing of the donor cell and enucleated oocyte membranes. The compound may be any compound capable of agglutinating cells. The compound may be a protein or glycoprotein capable of binding or agglutinating carbohydrate. More preferably the compound is a lectin. The lectin may be selected from the group including Concanavalin A, Canavalin A, Ricin, soybean lectin, lotus seed lectin and phytohemaglutinin (PHA). Preferably the compound is PHA.

In one preferred embodiment, the method of electrofusion described above also comprises a further fusion step, or the fusion step comprises described above comprises one donor progenitor cell, stem cell-like cell or MCT and two or more enucleated oocytes. The double fusion method has the advantageous effect of increasing the cytoplasmic volume of the reconstituted cell.

A reconstituted cell is typically activated by electrical and/or non-electrical means before, during, and/or after fusion of the nuclear donor and recipient oocyte (See, for example, Susko-Parrish et al., U.S. Pat. No. 5,496,720). Activation methods include:

1). Electric pulses;

2). Chemically induced shock;

3). Penetration by sperm;

4). Increasing levels of divalent cations in the oocyte by introducing divalent cations into the oocyte cytoplasm, e.g., magnesium, strontium, barium or calcium, e.g., in the form of an ionophore. Other methods of increasing divalent cation levels include the use of electric shock, treatment with ethanol and treatment with caged chelators; and

5). Reducing phosphorylation of cellular proteins in the oocyte by known methods, e.g., by the addition of kinase inhibitors, e.g., serine-threonine, kinase inhibitors, such as 6-dimethyl-aminopurine, staurosporine, 2-aminopurine, and sphingosine. Alternatively, phosphorylation of cellular proteins may be inhibited by introduction of a phosphatase into the oocyte, e.g., phosphatase 2A and phosphatase 2B.

The activated reconstituted cells, or embryo, are typically cultured in medium well known to those of ordinary skill in the art, and include, without limitation, TCM-199 plus 10% FSC, Tyrodes-Albumin-Lactate-Pyruvate (TALP), Ham's F-10 plus 10% FCS, synthetic oviductal fluid (“SOF”), B2, CRlaa, medium and high potassium simplex medium (“KSOM”).

The reconstituted cell may also be activated by known methods. Such methods include, e.g., culturing the reconstituted cell at sub-physiological temperature, in essence by applying a cold, or actually cool temperature shock to the reconstituted cell. This may be most conveniently done by culturing the reconstituted cell at room temperature, which is cold relative to the physiological temperature conditions to which embryos are normally exposed. Suitable oocyte activation methods are the subject of U.S. Pat. No. 5,496,720, to Susko-Parrish et al., herein incorporated by reference in its entirety.

The activated reconstituted cells may then be cultured in a suitable in vitro culture medium until the generation of cells and cell colonies. Culture media suitable for culturing and maturation of embryos are well known in the art. Examples of known media, which may be used for bovine embryo culture and maintenance, include Ham's F-10 plus 10% FCS, TCM-199 plus 10% FCS, Tyrodes-Albumin-Lactate-Pyruvate (TALP), Dulbecco's Phosphate Buffered Saline (PBS), Eagle's and Whitten's media. One of the most common media used for the collection and maturation of oocytes is TCM-199, and 1 to 20% serum supplement including fetal calf serum, newborn serum, estrual cow serum, lamb serum or steer serum. A preferred maintenance medium includes TCM-199 with Earl salts, 10% FSC, 0.2 mM Na pyruvate and 50 μg/ml gentamicin sulphate. Any of the above may also involve co-culture with a variety of cell types such as granulosa cells, oviduct cells, BRL cells and uterine cells and STO cells.

Afterward, the cultured reconstituted cell or embryos are preferably washed and then placed in a suitable media, e.g., TCM-199 medium containing 10% FCS contained in well plates which preferably contain a suitable confluent feeder layer. Suitable feeder layers include, by way of example, fibroblasts and epithelial cells, e.g., fibroblasts and uterine epithelial cells derived from ungulates, chicken fibroblasts, murine (e.g., mouse or rat) fibroblasts, STO and SI-m220 feeder cell lines, and BRL cells.

In some embodiments, the feeder cells comprise mouse embryonic fibroblasts. Preparation of a suitable fibroblast feeder layers are well known in the art.

The reconstituted cells are cultured on the feeder layer until the reconstituted cells reach a size suitable for transferring to a recipient female, or for obtaining cells which may be used to produce cells or cell colonies. Preferably, these reconstituted cells will be cultured until at least about 2 to 400 cells, more preferably about 4 to 128 cells, and most preferably at least about 50 cells. The culturing will be effected under suitable conditions, i.e., about 39° C. and 5% CO₂, with the culture medium changed in order to optimise growth typically about every 2-5 days, preferably about every 3 days.

The methods for embryo transfer and recipient animal management in the present invention are standard procedures used in the embryo transfer industry. Synchronous transfers are important for success of the present invention, i.e., the stage of the nuclear transfer embryo is in synchrony with the estrus cycle of the recipient female. This advantage and how to maintain recipients are reviewed in Siedel, G. E., Jr. (“Critical review of embryo transfer procedures with cattle” in Fertilization and Embryonic Development in vitro (1981) L. Mastroianni, Jr. and J. D. Biggers, ed., Plenum Press, New York, N.Y., page 323), the contents of which are hereby incorporated by reference.

Briefly, blastocysts may be transferred non-surgically or surgically into the uterus of a synchronized recipient. Other medium may also be employed using techniques and media well-known to those of ordinary skill in the art. In one procedure, cloned embryos are washed three times with fresh KSOM and cultured in KSOM with 0.1% BSA for 4 days and subsequently with 1% BSA for an additional 3 days, under 5% CO₂, 5% O₂ and 90% N₂ at 39° C. Embryo development is examined and graded by standard procedures known in the art. Cleavage rates are recorded on day 2 and cleaved embryos are cultured further for 7 days. On day seven, blastocyst development is recorded and one or two embryos, pending availability of embryos and/or animals, is transferred non-surgically into the uterus of each synchronized foster mother.

Foster mothers preferably are examined for pregnancy by rectal palpation or ultrasonography periodically, such as on days 40, 60, 90 and 120 of gestation. Careful observations and continuous ultrasound monitoring (monthly) preferably is made throughout pregnancy to evaluate embryonic loss at various stages of gestation. Any aborted fetuses should be harvested, if possible, for DNA typing to confirm clone status as well as routine pathological examinations.

The reconstituted cell, activated reconstituted cell, fetus and animal produced during the steps of such method, and cells, nuclei, and other cellular components which may be harvested therefrom, are also asserted as embodiments of the present invention. It is particularly preferred that the term animal produced be a viable animal.

The present invention can also be used to produce embryos, fetuses or offspring which can be used, for example, in cell, tissue and organ transplantation. By taking a fetal or adult cell from an animal and using it in the cloning procedure a variety of cells, tissues and possibly organs can be obtained from cloned fetuses as they develop through organogenesis. Cells, tissues, and organs can be isolated from cloned offspring as well. This process can provide a source of “materials” for many medical and veterinary therapies including cell and gene therapy. If the cells are transferred back into the animal in which the cells were derived, then immunological rejection is averted. Also, because many cell types can be isolated from these clones, other methodologies such as hematopoietic chimerism can be used to avoid immunological rejection among animals of the same species.

By “comprising” is meant including, but not limited to, whatever follows the word comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The invention will now be further described by way of reference only to the following non-limiting examples. It should be understood, however, that the examples following are illustrative only, and should not be taken in any way as a restriction on the generality of the invention described above. In particular, while the invention is described in detail in relation to the use of bovine and murine embryos, it will be clearly understood that the findings herein are not limited to these embryos, but would be useful growing stem cells or stem cell-like cells from embryos any animal including humans.

### Example 1

### Production of Bovine Stem Cells from Intact Embryo

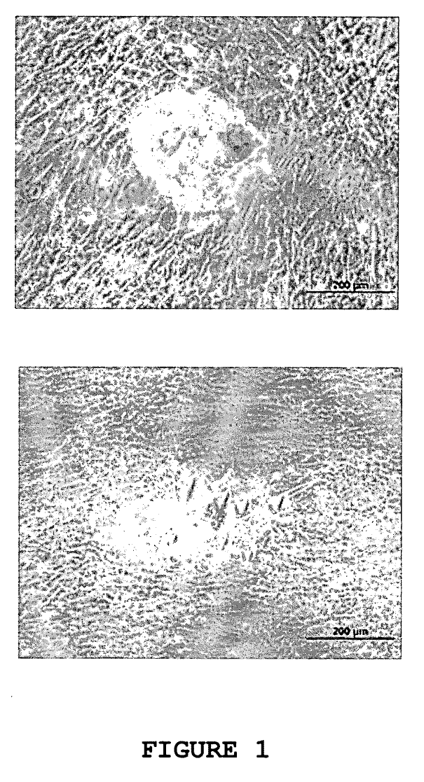

Bovine D7.5 embryos at the blastocyst stage were crushed and depressed into mouse embryonic feeder (MEF) layers, using fine-tipped glass micropipettes or Ultra-fine needle insulin syringe, and cultured in alpha-MEM medium in presence of bFGF, hEGF, ITS, hrLIF, 2-beta-ME, Glutamax, NEAA and 20% FCS. On the same day or on the following day the media was replaced with fresh media containing (treated group) or not containing (untreated group) 5 μM 5′-azacytidine. The media in each group was changed every 3 days. As seen in FIG. 1, both the untreated (Panel A) and treated (Panel B) are quite similar in appearance at the beginning of culture.

After seven to nine days, non-treated and treated “squashed” whole embryos developed morphologically distinguishable primary embryonal outgrowths.

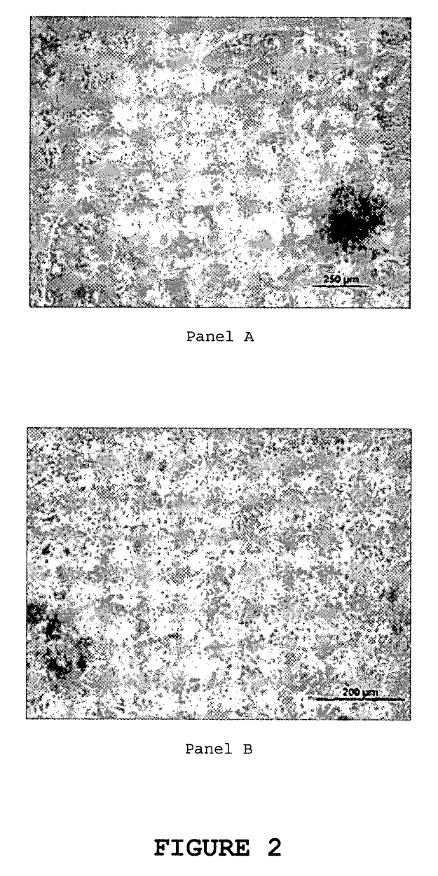

As shown in FIG. 2, untreated embryos formed outgrowths (control outgrowth, CO) consisting of pluripotent cells (PC), trophoblast cells, primitive endoderm cells and, possibly, primitive mesoderm (Panel A). Whereas treated embryos formed outgrowths (treated outgrowth, TRO), consisting only of cells with morphology, characteristic of bovine ES-like cells (Panel B). The size of outgrowths reached 0.6-0.8 cm in diameter.

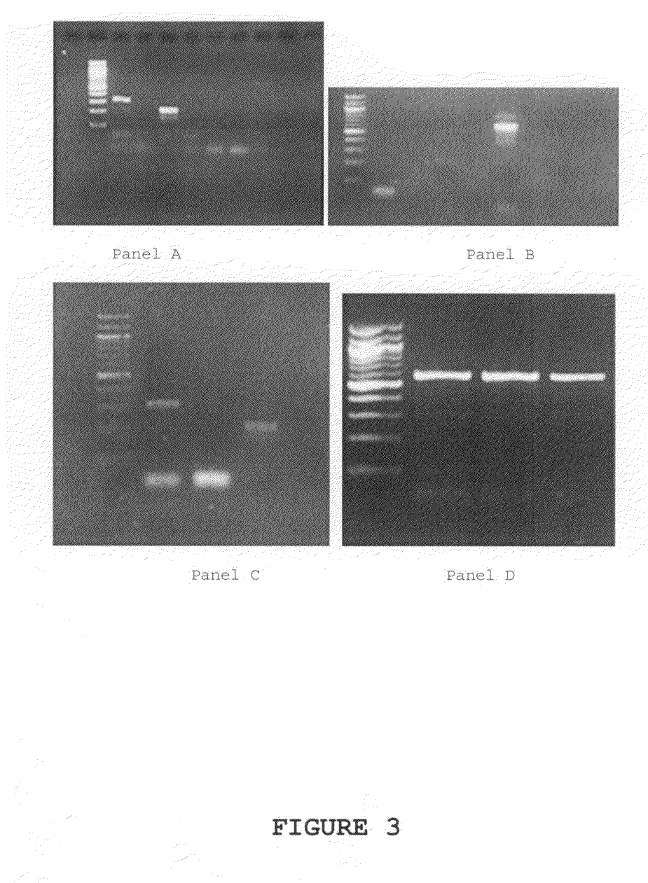

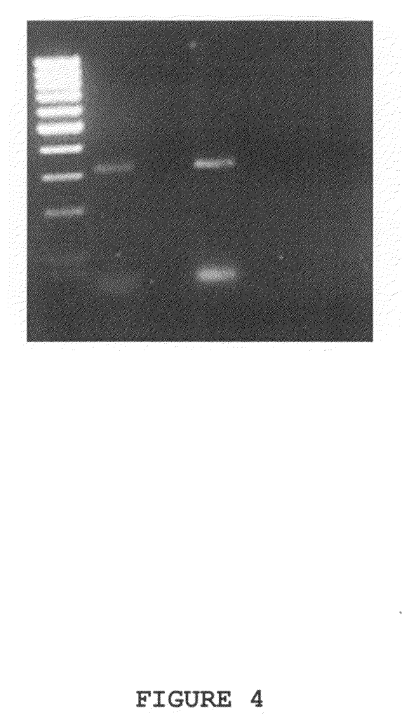

As shown in FIG. 3, only PC part of CO outgrowths expressed markers of pluripotency such as Oct4, Rex1 and SSEA-1 (Panels A, B). In TRO outgrowths all the cells expressed these markers (Panels C, D). Over the next 9-12 days the TRO outgrowths reached about 2 cm in diameter (filling the centre-well organ culture dish, BD, Cat. No 353001) without any changes in morphology. The outgrowths continued to express marker of pluripotency Oct4 (FIG. 4).

When PCs of CO outgrowths were mechanically dissected, cut into several pieces, passaged on fresh feeder layers and cultured in medium without 5′-azacytidine, they behaved in the same manner as normal primary cultures: they developed colonies, consisting of cells of the different cell types described previously. And only the PC component of these colonies expressed pluripotent markers.

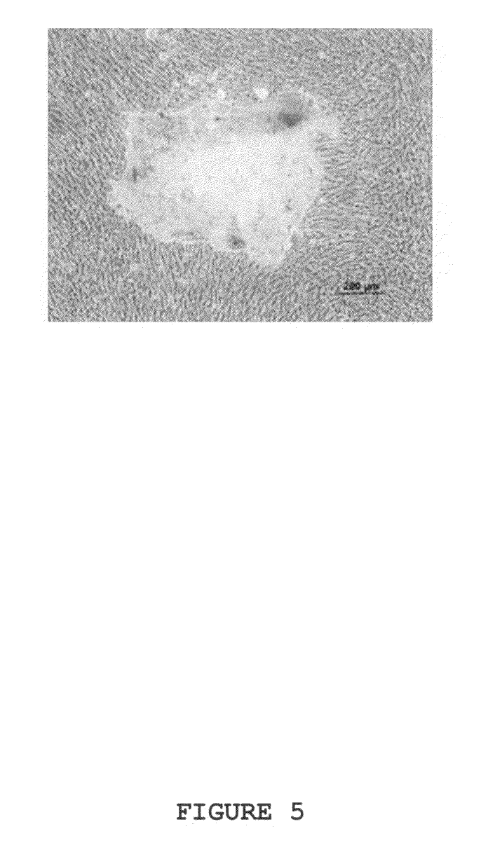

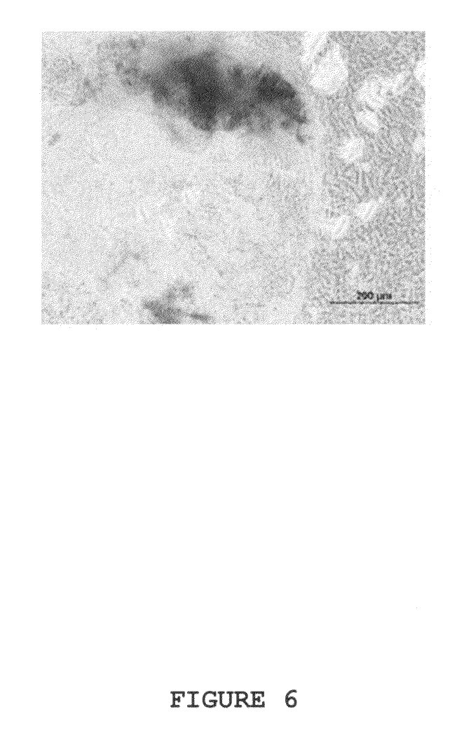

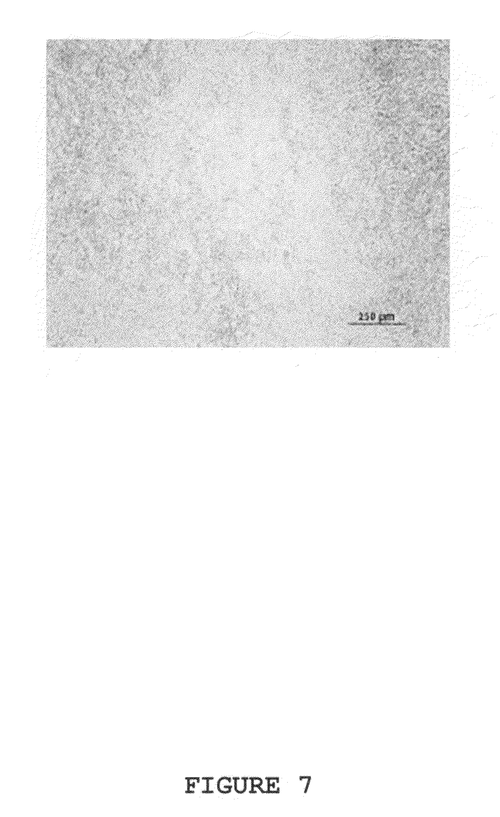

When primary TRO outgrowths, which were cultured for 14-21 days in the presence of 5′-azacytidine, were mechanically split into pieces of about 600-800 μm width, passaged onto fresh feeder layer and cultured in the presence of 5′-azacytidine they were able to form colonies of cells with uniform morphology, characteristic of bovine ES-like cells (FIGS. 5, 6 and 7). These cell colonies reached confluence in 14-21 days, depending on the number of pieces that were placed in the dish. The growing colonies which developed into confluent monolayers expressed marker of pluripotency, such as Oct4.

The same effects of 5′-azacytidine on primary and passaged cultures were not observed when the concentration of 5′-azacytidine was reduced to below 2.5 μM.

These data suggest:

1). ESCs can be isolated from whole embryo explants. A novel crushing technique results in greater efficiency in isolating ES-like primary outgrowths from embryos in vitro of primary blastocyst explants can increase the pluripotent component of a bovine blastocyst.
2. 5′-azacytidine treatment can induce more of the cells to remain pluripotent and induce proliferation of these cells as a uniform pluripotent population.
3. Continued culture of primary outgrowths in 5′-azacytidine results in maintenance of the cells in a pluripotent state.

### Example 2

### In Vivo Developmental Potential of Bovine ES Cells Isolated IN Example 1

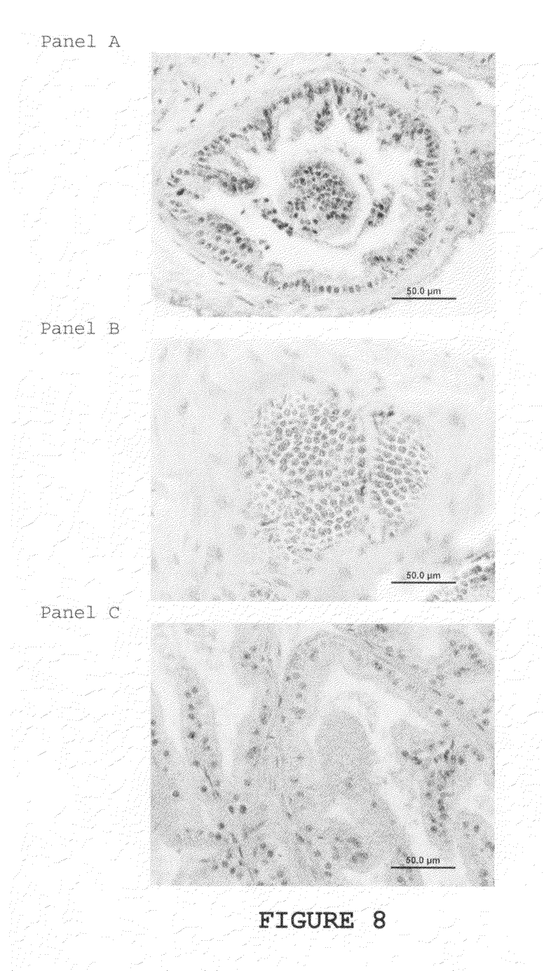

To investigate in vivo developmental potential of bovine ES cells and to examine their pluripotency, bovine ES cells, isolated and maintained in presence 5 μM 5′-azacytidine, were cut at passage 3 in to small pieces using a fine glass pipette. The small pieces consisted of 150-300 cells and these were then injected into testis's of SCID mice. After 8 weeks post-injection, teratomas partially expelled from testis's, were identified. Histochemical analysis confirmed the presence of derivatives of all three germ layers in developed teratomas (see FIG. 8 Panel A, B and D).

### Example 3

### Production of Mouse Stem Cells from Intact Embryo

To isolate ES cells from mouse embryos the techniques developed for bovine stem cells described in Example 1 was utilised.

Pre-pubertal (5-6 week old) 129sv females were super-ovulated using a routine protocol (1 IU PMSG and HCG injected i.p. 48h apart) before mating with OG2 males. The 129sv strain was chosen as it has been proven in the literature to facilitate isolation of ES cells. The OG2 males, used as studs for mating, were C57/B16 strain and transgenic for an Oct4-EGFP construct. All pluripotent cells from these mice express GFP and this property was exploited in this study with GFP being used as a fluorescent marker to facilitate the identification of ESCs once derived. Zygotes were collected from the oviduct of humanely killed female mice. Cumulus cells were removed from zygotes by treatment with Hyaluronidase (300 IU/ml) in KSOM medium (Chemicon). The zygotes were carefully washed to remove hyaluronidase and transferred to a fresh dish with culture drops in a 37° C. incubator and allowed to develop to the morula/blastocyst stage.

Treatment of Morula/Blastocyst with 1 μM of 5-aza-cytidine. Morula/blastocysts were cultured in the KSOM culture drops supplemented with 1 μM 5-aza-cytidine for 24 hr.

F1 (CBA/C57) MEF's were used as a feeder layer to facilitate attachment of embryos and subsequent isolation and support of ESCs. MEF's were inactivated by treatment with mitomycin C (10 μg/ml) for 2.5 hr in the 37° C. incubator and plated at the rate of 3×10⁵ cells/ml per 6 cm dish.

The zona pellucida was removed from embryos following a brief exposure to a 0.1% Acid Tyrode solution (pH2.8) and then washed 3 times in the culture medium (KSOM). Subsequently, they were pressed onto feeders using a 29 G needle (0.33 mm×12.7 mm). The medium was replaced with modified ESC medium i.e. DMEM supplemented with 1000×2-β-ME, 100×NEAA, 100× Glutamax, 20% Hyclone serum, 10 ng/ml mouse LIF, 10 ng/ml bFGF and 10 ng/ml hEGF, with 10 ng/ml Activin A, 10 ng/ml Nodal and 0.1 μM 5-aza-cytidine added freshly just prior to use. The dishes were then returned to the 37° C. incubator.

Media was replaced daily at 24 hr intervals. Just prior to changing media the cultures were observed and photographed for recording morphology (brightfield/Phase contrast) and GFP expression using an Olympus 1×71 Fluorescent microscope with the following: (1) Phase contrast (2) U-MNUA-2 (UV excitation) - - - 360-370 (excitation filters) 420-460 (emission filters), (3) U-MIGA 2 (Green excitation)—540-550 (excitation filters) 575-625 (emission filters) and (4) photographic attachments. The GFP expressing colonies were viewed under UV2A to determine whether the sample exhibited auto-fluorescence. Samples that expressed GFP and did not auto-fluoresce under UV2A were confirmed to be GFP positive.

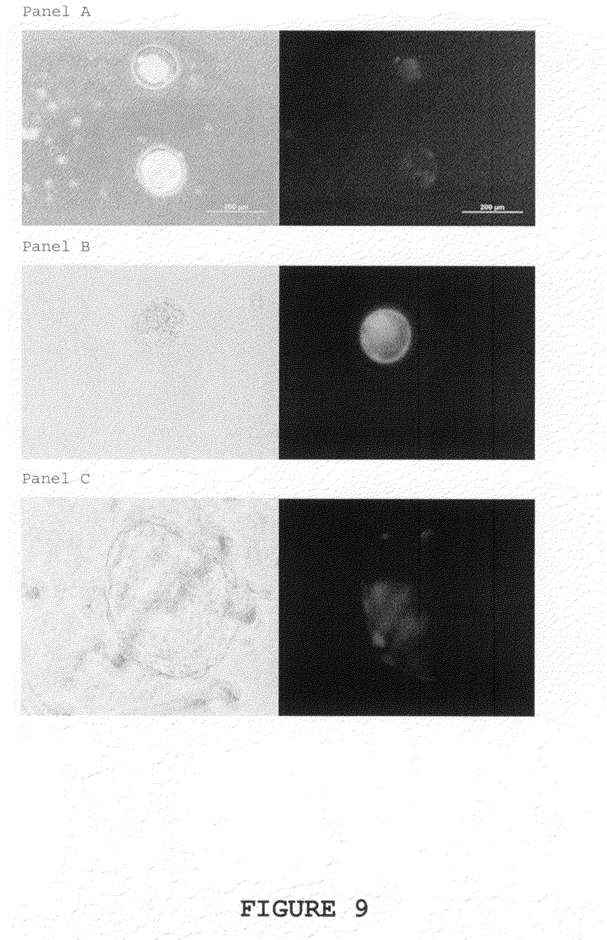

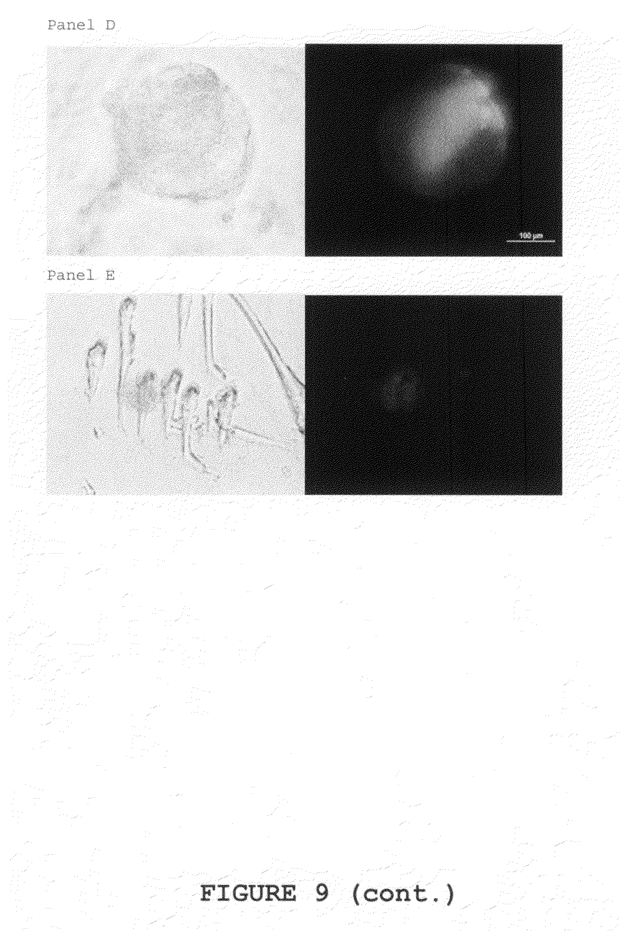

FIG. 9: OG2 F1 embryos treated as described in methods were observed and photographed under Brightfield, GFP and UV2A fluorescence microscopy to record morphology. GFP fluorescence was used to detect the presence (or lack thereof) of auto-fluorescence. In this regarded it should be noted that auto-fluorescence was not observed in any of the test samples (data not shown).

(Panel 9A) D0: Embryos (morula above and blastocyst below) in culture drops were observed prior to treatment with 5′ azacytidine. GFP expression can be visualised in the whole compact morula and predominantly in the inner cell mass (ICM) of the blastocyst.

(Panel 9B) D1: A blastocyst following a 24h treatment in KSOM culture medium, containing 1 μM 5-aza-cytidine (treated), and prior to removal of the zona and plating on MEF feeder layer.

(Panel 9C) D2: A treated blastocyst 24h after plating on a feeder layer in modified ESC medium.

(Panel 9D) D4: By D4 GFP expression was clearly visible in the blastocyst explant.

(Panel 9E) D9: By Day 9 the integrity of the embryo structure had broken down and ESC colonies were observed. Here one such colony expressing GFP is presented.

Pre-treatment of mouse pre-implantation embryos with a demethylation agent (5-aza-cytidine) for 24 h followed by removal of the zona pellucida and depressing of the intact embryo onto a feeder layer, resulted in expansion of embryonal explants. After a short period of culture (8-10 days) colonies of cells resembling ESCs were observed. Putative ESCs expressed a GFP transgene that was under the regulatory control of the pluripotent gene Oct4.

In summary we have described a novel method to isolate and establish, in culture, a population of ES like cells in the bovine and mouse. As the method of action of the chemicals used are not species specific we believe this invention has application in all vertebrate mammals, including human.

1. A method for producing stem cells or stem cell-like cells comprising:
(i) providing a culture comprising a mammalian embryo or embryo-derived mammalian cells on a feeder layer of cells;
(ii) introducing to said culture at least one demethylation agent; and
(iii) isolating pluripotent cells.

2. The method of claim 1, wherein the step of culturing the embryo or embryo-derived cells uses a culture medium selected from the group consisting of Synthetic Oviductal Fluid (SOF), Modified Eagle's Medium (MEM), Dulbecco's Modified Eagle's Medium (DMEM), RPMI 1640, F-12, IMDM, alpha-MEM and McCoy's Medium.

3. The method of claim 2, wherein the culture medium is alpha-MEM.

4. The method of claim 1, wherein the demethylation agent is 5-azacytidine, 5-aza-2′-deoxycytidine or ethionine.

5. The method of claim 1, wherein the embryo is crushed and depressed into the feeder layer.

6. The method of claim 1, wherein the embryo is obtained from a mammal selected from the group consisting of platypus, echidna, kangaroo, wallaby, shrews, moles, hedgehogs, tree shrews, elephant shrews, bats, primates (including chimpanzees, gorillas, orang-utans, humans), edentates, sloths, armadillos, anteaters, pangolins, rabbits, picas, rodents, whales, dolphins, porpoises, carnivores, aardvark, elephants, hyraxes, dugongs, manatees, horses, rhinos, tapirs, antelope, giraffe, cows or bulls, bison, buffalo, sheep, big-horn sheep, horses, ponies, donkeys, mule, deer, elk, caribou, goat, water buffalo, camels, llama, alpaca, pigs and hippos.

7. The method of claim 1, wherein the embryo is obtained from an ungulate selected from the group consisting of domestic or wild bovid, ovid, cervid, suid, equid and camelid.

8. The method of claim 1, wherein the embryo is obtained from a human subject.

9. An isolated stem cell or stem cell-like cell obtained by the method of claim 1.

10. A method for creating a normal non-human animal comprising the steps of:
(a) culturing a mammalian embryo or embryo-derived cells in the presence of a demethylation agent;
(b) isolating pluripotent cells;
(c) introducing said pluripotent cells into a blastocyst;
(d) implanting the blastocyst of (c) into a surrogate mother; and
(e) allowing the offspring to develop and be born.

11. The method of claim 10, wherein the animal is chimeric.

12. A composition comprising a population of stem cells and a culture medium, wherein the stem cells have been obtained by the method of claim 1.

13. A composition comprising a population of fully or partially purified progeny of the stem cells of claim 12.

14. The composition of claim 13, wherein the progeny have the capacity to be further differentiated.

15. A method for isolating and propagating pluripotent cells comprising the steps of:
(a) obtaining an embryo or embryo-like cells from a mammal;
(b) culturing said embryo or embryo-like cells in the presence of at least one demethylation agent;
(c) recovering said pluripotent cells; and
(d) culturing said pluripotent cells under expansion conditions to produce an expanded cell population.

16. An expanded cell population obtained by the method of claim 15.

17. A method for differentiating pluripotent cells ex vivo comprising the steps of:
(a) obtaining an embryo or embryo-like cells from a mammal;
(b) culturing said embryo or embryo-like cells in the presence of at least one demethylation agent;
(c) recovering said pluripotent cells;
(d) culturing said pluripotent cells under expansion conditions to produce an expanded cell population; and
(e) culturing the expanded cell population in the presence of desired differentiation factors.

18. The method of claim 17, wherein the differentiation factors are selected from the group consisting of basic fibroblast growth factor (bFGF); vascular endothelial growth factor (VEGF); dimethylsulfoxide (DMSO) and isoproterenol; and, fibroblast growth factor4 (FGF4) and hepatocyte growth factor (HGF).

19. A method for differentiating pluripotent cells in vivo comprising the steps of:
(a) obtaining an embryo or embryo-like cells from a mammal;
(b) culturing said embryo or embryo-like cells in the presence of at least one demethylation agent;
(c) recovering said pluripotent cells;
(d) culturing said pluripotent cells to produce an expanded cell population; and
(e) administering the expanded cell population to a mammalian host, wherein said cell population is engrafted and differentiated in vivo in tissue specific cells, such that the function of a cell or organ, defective due to injury, genetic disease, acquired disease or iatrogenic treatments, is augmented, reconstituted or provided for the first time.

20. The method of claim 19, wherein the tissue specific cells are of the osteoblast, chondrocyte, adipocyte, fibroblast, marrow stroma, skeletal muscle, smooth muscle, cardiac muscle, occular, endothelial, epithelial, hepatic, pancreatic, hematopoietic, glial, neuronal or oligodendrocyte cell type.

21. The method of claim 20, wherein the disease is selected from the group consisting of cancer, cardiovascular disease, metabolic disease, liver disease, diabetes, hepatitis, hemophilia, degenerative or traumatic neurological conditions, autoimmune disease, genetic deficiency, connective tissue disorders, anemia, infectious disease and transplant rejection.

22. A therapeutic composition comprising pluripotent cells and a pharmaceutically acceptable carrier, wherein the pluripotent cells are present in an amount effective to produce tissue selected from the group consisting of bone marrow, blood, spleen, liver, lung, intestinal tract, eye, brain, immune system, bone, connective tissue, muscle, heart, blood vessels, pancreas, central nervous system, kidney, bladder, skin, epithelial appendages, breast-mammary glands, fat tissue, and mucosal surfaces including oral esophageal, vaginal and anal and wherein said pluripotent cells are produced by culturing an embryo or embryo-derived cells in the presence of at least one demethylation agent.

23. A therapeutic method for restoring organ, tissue or cellular function to a mammalian animal in need thereof comprising the steps of:
(a) obtaining an embryo or embryo-like cells from a mammal;
(b) culturing said embryo or embryo-like cells in the presence of at least one demethylation agent;
(c) recovering said pluripotent cells; and
(d) administering the pluripotent cells to the mammalian animal, wherein organ, tissue or cellular function is restored.
